(19) United States
(12) Patent Application Publication
(10) Pub. No.: US 2024/0424209 A1
(43) Pub. Date: Dec. 26, 2024

(54) SYSTEMS AND METHODS FOR DETERMINING PATTERNS IN AN INSULIN DELIVERY SYSTEM TO IMPROVE USER EXPERIENCE

(71) Applicant: Tandem Diabetes Care, Inc., San Diego, CA (US)

(72) Inventors: Alexandra Elena CONSTANTIN, San Diego, CA (US); Alex TRAHAN, San Diego, CA (US); Lars MUELLER, San Diego, CA (US); Zahra YARMOHAMMADI, San Diego, CA (US); Gina MERCHANT, Encinitas, CA (US); Daphna ZEILINGOLD, San Diego, CA (US)

(21) Appl. No.: 18/749,341

(22) Filed: Jun. 20, 2024

Related U.S. Application Data
(60) Provisional application No. 63/510,067, filed on Jun. 23, 2023.

(51) Int. Cl. A61M 5/172; G16H 20/17

(52) U.S. Cl. CPC A61M 5/1723; G16H 20/17; A61M 2205/502; A61M 2205/583; A61M 2230/201

(57) ABSTRACT

Systems and methods are provided for determining patterns, trends, anomalies, and/or abnormalities in data relating to operation of an insulin delivery pump and user outcomes relating to an insulin delivery pump and determining certain actions and/or operational adjustments to improve the user outcomes, such as blood glucose levels. For example, troubleshooting actions may be recommended upon determining a pattern, trend, abnormality, and/or anomaly in blood glucose levels that is undesirable. In another example, operation of the insulin delivery pump may be adjusted based habits and/or behaviors of the user. For example, insulin delivery timing may be adjusted based on exercise, activity and/or eating patterns. Based on the detected patterns, trends, anomalies, and/or abnormalities, a user device such as a mobile phone or smart device may present prompts for more information, alerts, status updates, and other information intended to improve the user experience.

### CROSS-REFERENCE TO RELATED APPLICATION

This application claims priority to U.S. Patent Application Ser. No. 63/510,067, filed on Jun. 23, 2023, the entire contents of which is incorporated herein by reference.

### TECHNICAL FIELD

This technology generally relates to the field of medication delivery pumps including pumps for delivering insulin to a user. For example, systems and methods are provided herein for improving a user experience for a user of an insulin delivery pump.

### BACKGROUND

Nearly 1 in 10 individuals in the United States are affected by diabetes. As technology advances, techniques for glucose monitoring and even insulin delivery so too develop and evolve. To manage the condition, historically many of these individuals were required to administer a blood draw, for example a need prick in the fingertip to analyze the blood to determine blood glucose levels. If the blood glucose level did not satisfy a threshold, the individual may have to administer an insulin shot, meaning an injection of insulin into the subcutaneous tissue using a needle and syringe.

With advances in monitoring technology, continuous glucose monitoring using a wearable patch including a small needle provides on demand glucose monitoring without the need for multiple needle pricks throughout the day. Additionally, technological advances in pump technology and insulin administration have resulted in wearable pumps and even patch pumps adhered to a user. Using the blood glucose monitor, the physiological effect of insulin delivered to the user may be evaluated to determine the effectiveness of the insulin dosing an timing. Other smart devices including wearable devices may collect information relevant to insulin delivery and blood glucose levels such as a smartphone, pedometer, heart rate sensor, temperature sensor, and the like.

While the these advancements in technology have enable users and system operators to collect vast amounts of data including biometric data, blood glucose data, insulin delivery information, and pump operation data, processing the data and applying the information in a meaningful way presents many challenges due to the large volume of the data and the complexities involved in adjusting insulin pump operation, insulin dosing, and user behavior to improve the user experience and/or physiological outcomes such as blood glucose levels.

Accordingly, there is a need for improved systems and methods for adjusting pump operation and/or user behavior to improve the user experience relating to an insulin delivery pump.

### SUMMARY

Provided herein are systems and methods for determining patterns, trends, anomalies, and/or abnormalities in data relating to operation of an insulin delivery pump and/or in user outcomes relating to the insulin delivery pump. Certain actions and/or operational adjustments may be determined from the patterns, trends, anomalies, and/or abnormalities to improve the user outcomes, such as blood glucose levels, and/or the user experience. For example, troubleshooting actions may be recommended upon determining a pattern, trend, abnormality, and/or anomaly in blood glucose levels that is undesirable. In another example, operation of the insulin delivery pump may be adjusted based on habits and/or behaviors of the user. For example, insulin delivery timing may be adjusted based on exercise, activity, and/or eating patterns. Based on the detected patterns, trends, anomalies, and/or abnormalities, a user device such as a mobile phone or smart device may present prompts for more information, alerts, status updates, and other information intended to improve the user experience.

Systems and methods are provided for determining patterns in an insulin delivery system to improve a user's glucose levels over time. Such systems and methods may include determining user data corresponding to operational information of an insulin pump of the insulin delivery system and/or user behavior information, determining first glucose data corresponding to a set of blood glucose values of a user of the insulin pump over a first period of time, determining an abnormality in the first glucose data based on the glucose data exceeding a threshold value for a second period of time within the first period of time, determining an action associated with the abnormality and the user data, causing a display to present a message indicative of the action, determining second glucose data over a third period of time after the first period of time and after causing the display to present the message, and determining the second glucose data satisfies the threshold value for a fourth time period during the third period of time, the second glucose data indicative of improved glucose levels for the user.

The systems and methods may further include determining the user data corresponds to the second period of time. The user data may be indicative of exercise data, infusion site data, insulin delivery data, and/or food data. The first glucose data and the second glucose data may be generated by a blood glucose monitor. The action may correspond to eating food. The action corresponds to adjusting operational settings of the insulin pump, and/or removing the insulin pump from the user. The second period of time may be the same amount as the fourth period of time.

The systems and methods may include determining to request additional user data based on the user data and/or the abnormality, and determining prompt information to elicit the additional user data based on the user data and/or the abnormality. The systems and methods may include causing the display to present the prompt information, and determining the additional user data after the causing the display to present the prompt information. The additional user data may correspond to additional exercise data, additional infusion site data, additional insulin delivery data, and/or additional food data. Determining the action may be based on the additional user data.

In yet another example, systems and methods are provided for detecting patterns relating to food intake in an insulin delivery system to improve a user's glucose levels over time. The systems and methods may include determining biometric data corresponding to a user of the insulin delivery system, the insulin delivery system including an insulin pump worn by the user, determining a first basal rate, first carb ratio, first insulin sensitivity value, and/or first meal value corresponding to the user based on the biometric data, determining initial insulin delivery parameters for the insulin pump based on the first basal rate, first carb ratio, first insulin sensitivity value, and/or first meal value, determining estimated glucose range over a first period of time based on the first basal rate, first carb ratio, first insulin sensitivity value, and/or first meal value, determining actual glucose data over a first period of time, the actual glucose data corresponding to the initial insulin delivery parameters, determining the actual glucose data exceeds the estimated glucose range, and causing a user device to present a request for food data based on the actual glucose data exceeding the estimated glucose range.

The systems and methods may further include determining initial food data. Determining a first basal rate, a first carb ratio, a first insulin sensitivity value, and/or a first meal value may further be based on the initial food data. The initial food data may include timestamps indicative of times associated with food consumption by the user. The systems and methods may further include associating the first meal value with each time timestamp of the timestamps.

The systems and methods may further include receiving the food data. The food data may include timestamps indicative of times associated with food consumption by the user. The systems and methods may further include determining a second basal rate, second carb ratio, second insulin sensitivity value, and/or second meal value corresponding to the food data and actual glucose data and/or biometric data. The systems and methods may further include determining adjusted insulin delivery parameters based on the second basal rate, second carb ratio, second insulin sensitivity value, and/or second meal value. The food data may be audio data. The systems and methods may further include causing a user device to present the request for food data including presenting the request for food data on a display of the user device. The systems and methods may further include presenting three meal size options on a display of the user device.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

FIG. 1 illustrates an exemplary insulin pump system including a wearable insulin pump and periphery and remote devices, in accordance with some aspects.

FIG. 2 illustrates an exemplary data flow depicting determining a pattern in data corresponding to an insulin pump and determining actions corresponding to the detected patterns.

FIG. 3 illustrates an exemplary data flow for generating a pattern alert with a reminder prompt.

FIG. 4A illustrates exemplary user interfaces including prompts for troubleshooting when an issue is detected.

FIG. 4B illustrates exemplary user interfaces including prompts for collecting food data.

FIG. 5 illustrates an exemplary data flow for comparing glucose levels to expected levels and improving blood glucose levels based on food data.

FIG. 6 illustrates a schematic block diagram of a remote device in accordance with one or more exemplary embodiments of the disclosure.

The foregoing and other features of the present invention will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

The present disclosure is directed to systems and methods for an insulin pump system including an insulin pump for detecting patterns, trends, abnormalities, and/or anomalies and determining actions for improving the user experience. For example an insulin pump may be used by and worn by a user. The insulin pump may periodically deliver insulin to the user based on certain pump parameters (e.g., settings, rules, and/or constraints). The insulin pump may include a processor for operating the pump to achieve desired insulin delivery. The pump parameters and insulin delivery may be monitored over time in view of other data may be collected regarding the user (e.g., physiological outcomes such as blood glucose levels) and/or user behavior. Trends in the data relating to insulin delivery and physiological outcomes, for example, may be detected to guide and/or inform future pump operation and user behavior for improving pump operation and/or physiological outcomes.

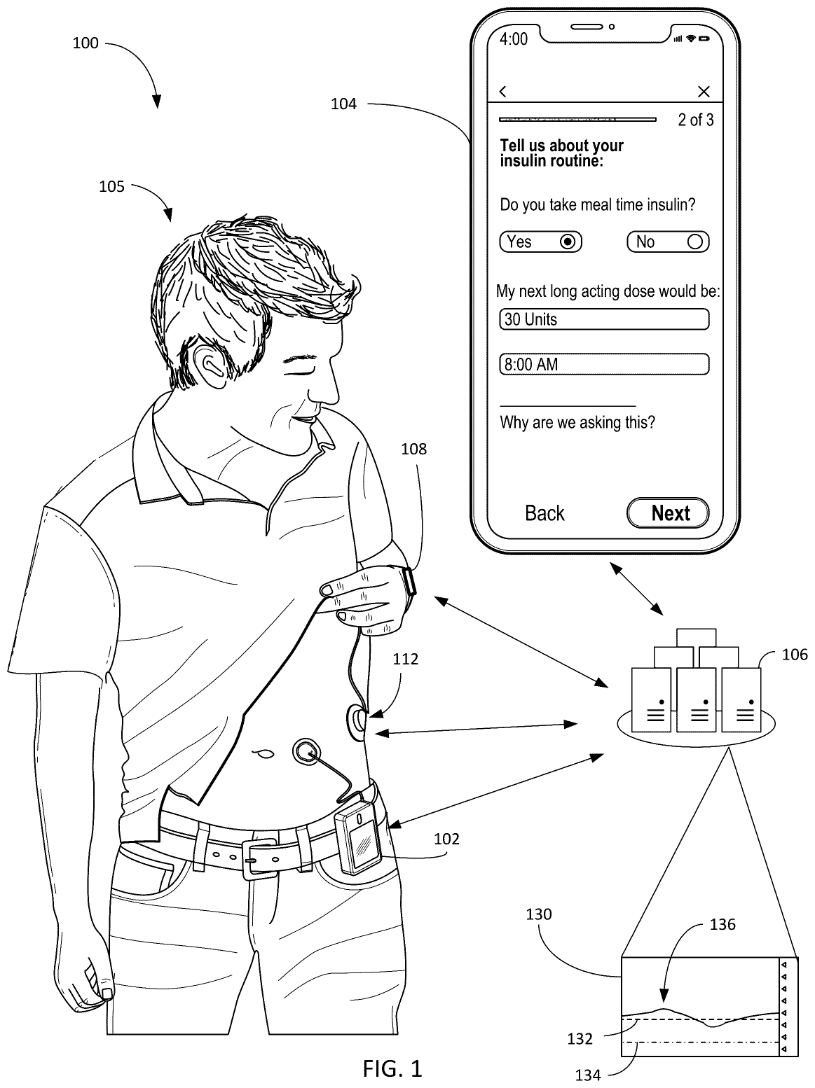

Referring now to FIG. 1, an insulin pump system including a wearable pump and periphery and remote devices is illustrated. Insulin pump system 100 may include insulin pump 102, mobile device 104, and/or remote device 106. Insulin pump system 100 may communicate with other devices such as glucose monitor 112, smart device 108 and/or other computing devices such as a healthcare provider device. Insulin pump system 100 may include greater or fewer devices than those illustrated in FIG. 1 and/or one or more device in FIG. 1 may be optional.

Insulin pump 102 may include pump housing 118 which may include a display and may house a pump designed to pump insulin into a user (e.g., user 105). For example, insulin pump 102 may include a delivery component which may include tubing and/or a delivery patch having a needle or cannula for delivering insulin into a subcutaneous area of the user at an infusion site. Insulin pump 102 may include a compartment for holding a volume of insulin and may selectively deliver an amount of insulin (e.g., a bolus) to the user via the pump and delivery component. While insulin pump 120 is described herein as an insulin pump, it is understood that insulin pump 120 may alternatively be any fluid pump for pumping medication or other fluids (e.g., saline) into the body of a user.

Insulin pump 102 may, in one example, be the same as or similar to a t:slim X2™ insulin pump available from Tandem Diabetes Care, Inc. of San Diego, California and/or the insulin pump described in U.S. Patent App. Pub. No. 2014/0276423, published on Sep. 18, 2014 and assigned to Tandem Diabetes Care, Inc., and/or the insulin pump described in U.S. Patent App. Pub. No. 2011/014461, published on Jun. 16, 2011 and assigned to Tandem Diabetes Care, Inc., each of which are hereby incorporated by reference in their entirety. However, it is understood that insulin pump 102 may be any type of insulin pump for delivering insulin to the user and having the functionality described herein.

Insulin pump 102 may include one or more processors and memory as well as a communication chip for communicating with one or more periphery devices and/or remote devices (e.g., remote device 106). For example, insulin pump 102 may communicate with mobile device 104 and/or smart device 108 via a suitable wireless connection (e.g., Bluetooth, Bluetooth Low Energy (BLE), Wi-Fi Direct, any other near field communication protocol, and/or the like). Insulin pump 102 may optionally communicate directly with remote device 106, glucose monitoring sensor 112 and/or any other devices.

As shown in FIG. 1, insulin pump 102 may be an extracorporeal and/or ambulatory and may be worn by the user. For example, insulin pump 102 may be worn on the user's belt or waist band. In another example, insulin pump 102 may be worn on a user's arm (e.g., using an arm band). Alternatively, insulin pump 102 may be a patch pump and/or may be adhered to the user's skin. The user may also have one or more periphery devices nearby. For example, the user may hold mobile device 104 in their pocket or personal bag (e.g., backpack or purse) and/or may wear smart device 108 (e.g., on their wrist). Due to the proximity of the periphery devices to insulin pump 102, insulin pump 102 may use short range communication technology (e.g., Wi-Fi, cellular network, Bluetooth, Bluetooth Low Energy (BLE), Wi-Fi Direct, near field communication protocol, and the like) for communication with the periphery devices.

Mobile device 104 may be any suitable mobile device such as a mobile phone (e.g., smart phone), tablet, laptop, wearable, charging device, smart device, and/or smart sensor. Mobile device 104 may include a computer processor, memory, and/or a communication unit which may facilitate communication between mobile device and insulin pump 102, remote device 106, smart device 108, and/or any other devices (e.g., healthcare provider device, smart sensors such as glucose monitoring sensor 112, and/or any other devices).

Mobile device 104 may communicate with any devices in insulin pump system 100 and any other devices via any suitable wired or wireless system (e.g., Wi-Fi, cellular network, Bluetooth, Bluetooth Low Energy (BLE), near field communication protocol, etc.). Mobile device 104 may support communication with various devices over different communication networks. For example, mobile device 104 may communicate with insulin pump 102 via one network type (e.g., a near field communication network or Bluetooth) and may communicate with remote device 106 via a different network type (e.g., cellular and/or Wi-Fi protocols).

Remote device 106 may be any computing device having a processor, memory and a communication unit. For example, remote device 106 may be one or more servers, datastores, or the like. Remote device 106 may communicate with any devices in insulin pump system 100 and any other devices via any well-known wired or wireless system (e.g., Wi-Fi, cellular network, Bluetooth, Bluetooth Low Energy (BLE), near field communication protocol, etc.).

Insulin pump 102, mobile device, 104, remote device 106, and/or smart device 108 may be in communication with glucose monitor 112, which may be any suitable continuous glucose monitoring sensor such as the DexCom G7 Continuous Glucose Monitoring (CGM) system available from DexCom, Inc. of San Diego, California and/or other sensor device. For example, insulin pump 102, mobile device 104, remote device 104, and/or smart device 108 may receive blood glucose data (e.g., glucose measurements) and/or other health related information from glucose monitor 112 and may share such information with other devices in insulin pump system 100.

Insulin pump 102 may be used by the user to selectively deliver an amount of insulin (e.g., a bolus) to regulate the user's blood glucose levels. Insulin pump 102 may be programmed to deliver insulin based on certain rules and/or constraints (e.g., at certain times and/or volumes). Additionally, or alternatively, the insulin pump may deliver insulin based on input data received and/or determined (e.g., via user input and/or sensor data).

Insulin pump system 100 may process data generated by the system and may determine actions and/or adjustments for improving outcomes such as physiological outcomes (e.g., blood glucose levels) and/or ease of use for improving the user experience of insulin pump 102. For example, insulin pump 102 may send pump operation data (e.g., operational data) to remote device 106, glucose monitor 112 may share blood glucose data to remote device 106, and/or smart device 108 may share behavioral data (e.g., steps, activity or exercise information, sleep information, or the like) and/or physiological information (e.g., heart rate data, electrocardiogram (ECG) information, temperature information, or the like) with remote device 106.

Mobile device 104, which may be a user device such as a smart phone, may be used by the user (e.g. user 105) to input certain information about the user, the user's behavior, activity, habits, medical information, biometric information, and any other relevant user information. For example, as shown in FIG. 1, a user of user device 104 may input information about whether they typically take meal time insulin, dosing, timing for insulin and other information about the user's meal time routine. Other information such as biometric information, medical history, user's history and the like may be input into mobile device 104 during an onboarding step or periodically during use of insulin pump system 100.

Data generated by or otherwise received from insulin pump 102, glucose monitor 112, smart device 108, mobile device 104 and/or other devices in or in communication with insulin pump system 100 may be processed and/or analyzed to determine patterns, trends, anomalies, and/or abnormalities for improving physiological outcomes and/or ease of use of insulin pump 102. For example, such data may be processed and/or analyzed by remote device 106 to determine trends or patterns in blood glucose levels and may present the user with actionable insight for adjusting user behavior and/or pump operation to improve blood glucose levels. Optionally, machine learning algorithms (e.g., neural networks) may be trained and used to detect such trends, patterns, anomalies, abnormalities, etc. In one example, remote device 106 may cause mobile device to present questions or other information for troubleshooting and/or optimizing or improving operation of insulin pump 102.

As shown in FIG. 1, remote device remote device 106 may analyze glucose levels 136 over time and determine if and for how long glucose levels exceed certain thresholds. For example, upper threshold 132 and lower 134 may define a “normal range” and blood glucose measurements (e.g., from glucose device 112) may be considered outside of normal range. Remote device 106 may consider what activities and behavior occurred, or did not occur (e.g., eating) during the time frame for which blood glucose levels were out of range or in range and may make recommendations for the user based on these associations.

If the user is achieving positive physiological outcomes (e.g., pattern of blood glucose levels within the normal range) then remote device 106 may inform the user (e.g., via mobile device 104 and/or smart device 108) to maintain the same regime (e.g., pump parameters and/or behavior). If the user is not achieving positive outcomes, remote device 106 may inform the user and may either request more information, suggest that the user periodically provide more information, suggest behavior actions (e.g., to eat), and/or suggest a change in insulin pump operation. In some cases, remote device may suggest that insulin pump 102 be removed and/or infusion site relocated.

The user data may also or alternatively be compared, to the extent permitted under law, to data of other users (e.g., anonymized data), such as other users of the insulin pump, users of the same health care provider. Alternatively, or additionally, anonymized user data may be used to for comparison purposes (e.g., to the extent permitted under law) to other user's data to inform decisions, analysis, and/or recommendations for such other users.

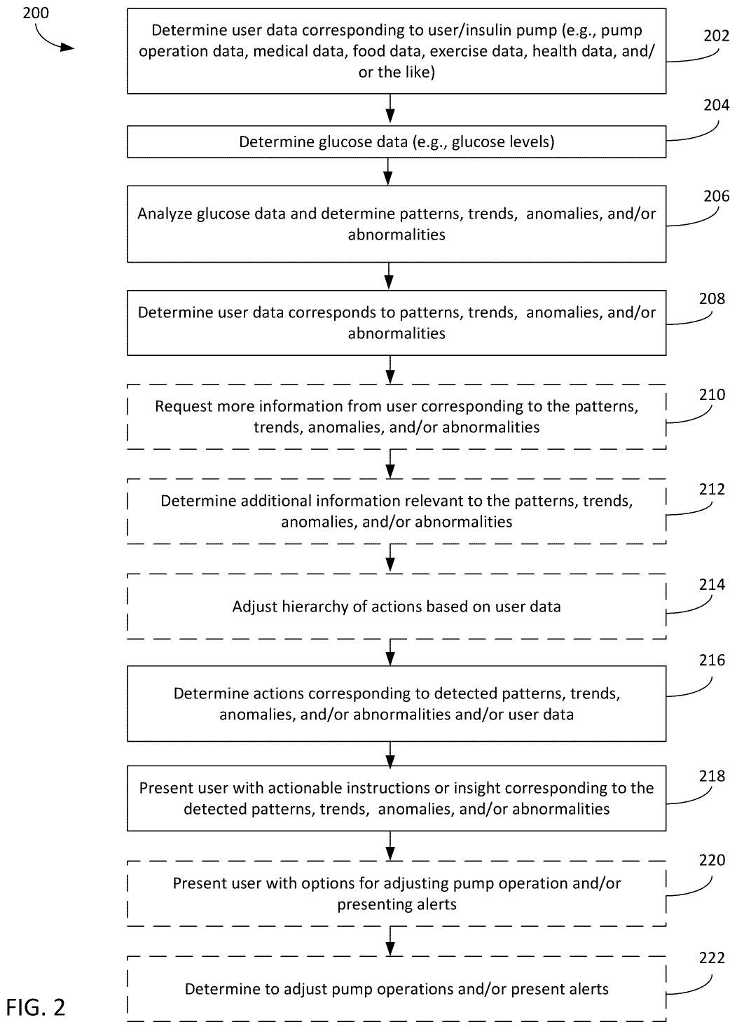

Referring now to FIG. 2, an exemplary process flow depicting determining a pattern in data corresponding to an insulin pump and determining actions corresponding to the detected patterns. One or more steps of process flow 200 may be performed by remote device (e.g., remote device 106 of FIG. 1) and/or a periphery device (e.g., mobile device 104 and/or smart device 108 of FIG. 1). It is further understood that one or more steps in FIG. 2 may be optional and/or additional or fewer steps than those shown in FIG. 2 may be performed.

To initiate process flow 200, at block 202, computer-executable instructions stored on a memory of a device, such as a remote device and/or a periphery device, may be executed determine user data corresponding to information about the user and/or information about the insulin pump. For example, user data may include biometric data, physiological data, medical information, medical history information, food data (e.g., information about a size, quantity, time of a meal, etc.), behavior data, exercise data, health data, and/or any other data relevant to the insulin pump system.

At block 204, computer-executable instructions stored on a memory of a device, such as a remote device and/or a periphery device, may be executed to determine glucose data (e.g., from a glucose device), which may include blood glucose measurements over time indicative of blood glucose levels. At block 206, computer-executable instructions stored on a memory of a device, such as a remote device and/or a periphery device, may be executed to analyze the glucose data and determine patterns, trends, anomalies, and/or abnormalities. For example, upper and lower thresholds may define a normal range and measurements outside of the normal range may be determined.

At block 208, computer-executable instructions stored on a memory of a device, such as a remote device and/or a periphery device, may be executed to determine the user data corresponds to the patterns, trends, anomalies, and/or abnormalities detected at block 206. For example, the time for which a pattern, trend, anomaly, and/or abnormality occurred may be determined and user data occurring at that same time or related to that time, may be determined and associated with the patterns, trends, anomalies, and/or abnormalities. Alternatively, a lack of certain user data occurring at that same time may also or alternatively be relevant (e.g., lack of activity or food consumption).

At optional block 210, computer-executable instructions stored on a memory of a device, such as a remote device and/or a periphery device, may be executed to request more information from the user corresponding to or relating to the pattern, trend, anomaly, and/or abnormality detected and/or the user data. For example, a periphery device may prompt the user for more information.

At optional block 212, computer-executable instructions stored on a memory of a device, such as a remote device and/or a periphery device, may be executed to determine and/or receive such additional information. The additional information may include additional user information such as biometric data, physiological data, medical information, medical history information, food data (e.g., information about a size, quantity, time of a meal, etc.), behavior data, exercise data, health data, and/or any other data relevant to the insulin pump system

At optional block 214, computer-executable instructions stored on a memory of a device, such as a remote device and/or a periphery device, may be executed to adjust the hierarchy of available actions to take based on the user's history, user's past compliance, past pump operations, and/or any other relevant data. In one example, machine learning may optionally be used to determine and/or adjust the hierarchy of available actions based on the user's history, user's past compliance, past pump operations, and/or any other relevant data.

At block 216, computer-executable instructions stored on a memory of a device, such as a remote device and/or a periphery device, may be executed to determine actions that correspond to detected patterns, trends, anomalies, and/or abnormalities and/or user data detected. For example, a database of actions associated with certain patents, trends, anomalies, and/or user data detected may be consulted to determine appropriate action.

In one example, consistent high glucose levels may correspond to the actions of adjusting an amount of insulin delivered, suggesting that the user more routinely enter food data, recommending that the user relocate the infusion site for the insulin pump, suggesting that the user turn on reminders for inputting exercise and/or food events, and the like. The actions may be troubleshooting actions, positive reinforcement prompts, reminder recommendations, food data prompts, and any other relevant actions for improving operation and/or physiological outcomes relating to the insulin delivery system.

At optional block 218, computer-executable instructions stored on a memory of a device, such as a remote device and/or a periphery device, may be executed to present the user with the determined action (e.g., actionable instructions, prompt, or other message or insight) corresponding to the detected patterns, trends, anomalies, and/or abnormalities. At optional block 220, computer-executable instructions stored on a memory of a device, such as a remote device and/or a periphery device, may be executed to present the user with option to adjust pump operation (e.g., based on an adjusted basal rate, carb ratio, insulation sensitivity level, food data, or the like) and/or to turn on alerts or reminders.

At optional block 222, computer-executable instructions stored on a memory of a device, such as a remote device and/or a periphery device, may be executed to determine to adjust pump operations and/or present alerts at a certain frequency based on input received from the user.

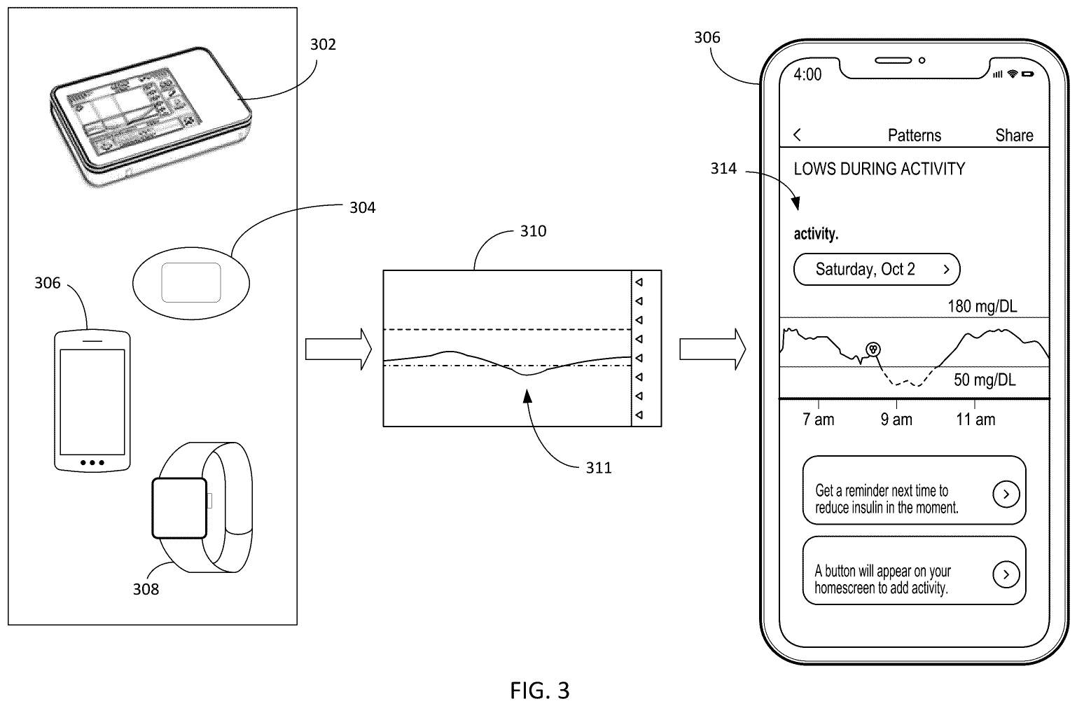

Referring now to FIG. 3, an exemplary data flow for generating an alert with a reminder prompt is illustrated. As shown in FIG. 3, data may be generated by insulin device 302 (e.g., insulin device 102 of FIG. 1), glucose monitor 304 (e.g., glucose monitor 112 of FIG. 1), mobile device 306 (e.g., mobile device 104 of FIG. 1) and/or smart device 308 (e.g., smart device 108 of FIG. 1). Such data may be processed or analyzed by a remote device (e.g., remote device 106 of FIG. 1) to detect patterns, trends, abnormalities, and/or anomalies.

For example, glucose plot 310 may be generated to determine glucose measurements over time, and may associate certain user data (e.g., behavioral data) with patterns, trends, abnormalities, and/or anomalies in such plot. For example, lows in glucose levels (e.g., low 311) may be determined and associated with exercise by the user. Mobile device 306 may then present a user interface, indicating that lows during activity (e.g., exercise) were detected and may even present a plot showing such lows, which may include the time. The interface may also ask the user if they would like to turn on a reminder next time there is a low and/or may inform the user about where to add activity data.

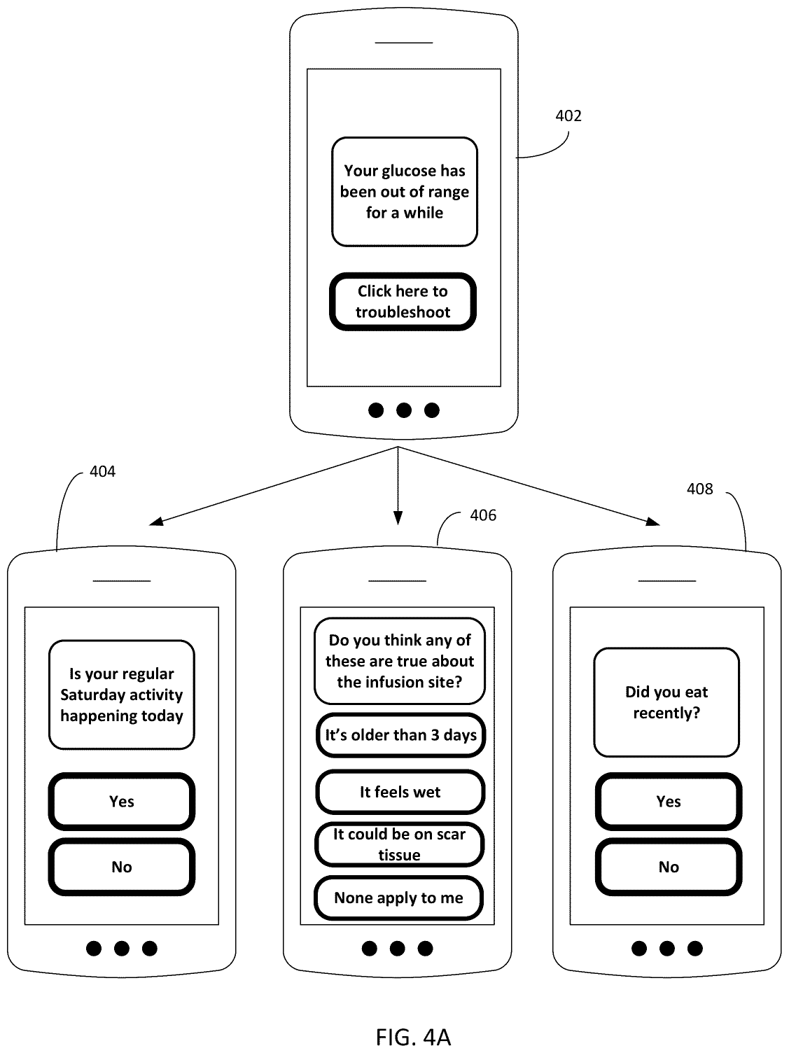

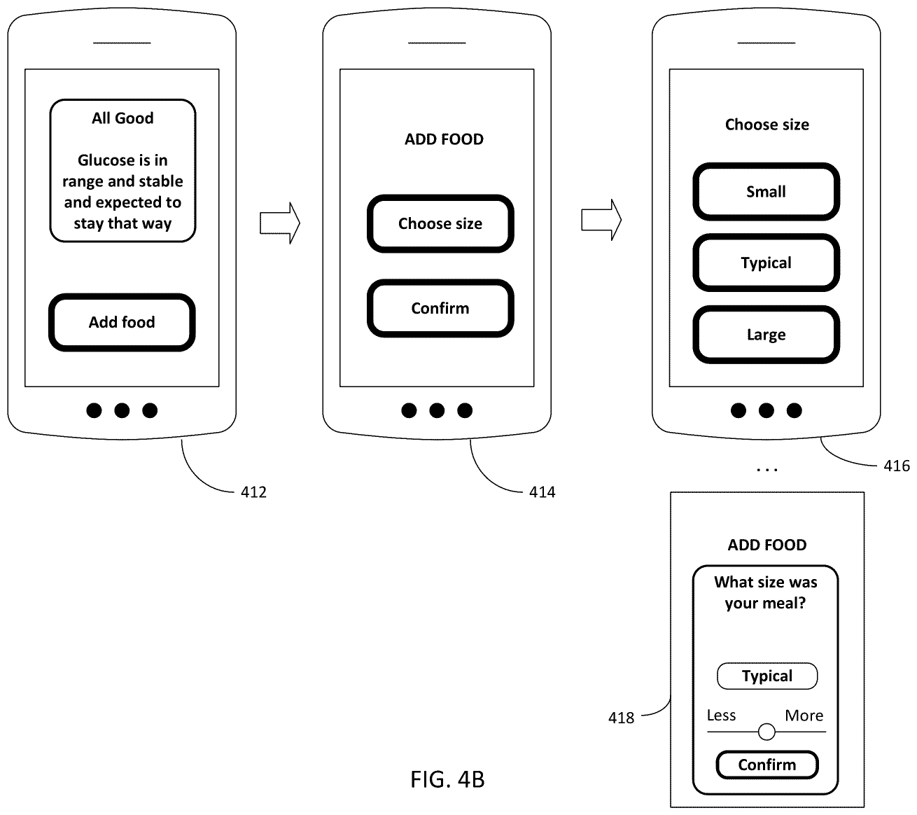

Referring now to FIG. 4A, exemplary user interfaces including prompts for troubleshooting when an issue is detected are illustrated. The issue may be persistent low blood glucose levels over a prolonged period of time, for example. For example, upon detecting the issue, mobile device 402 (e.g., mobile device 104 of FIG. 1) may be caused (e.g., by a remote device) to present an alert for the user, indicating that, for example, the user's blood sugar is too low. Mobile device 402 may also present an inquiry whether the user would like to troubleshoot, which may be a digital button or other engageable feature.

If the user engages the button or otherwise indicates that the user would like to troubleshoot the detected issue, the system may determine an associated action or prompt based on the detected issue and/or other user data. For example, if the user data indicates that that the issue may be related to exercise or activity, prompt 404 may be caused to be presented and may ask if the user has done or will do their regular activity for that day. The prompt may include engageable features for answering the question.

Alternatively, if the issue is determined to be related to pump hardware connection to the user's body, prompt 406 may inquire whether certain possibilities are true with respect to the infusion site. For example, the prompt may ask if the infusion site is older than 3 days, feels wet, and/or could be on scar tissue. Based on this prompt, actionable insight and/or recommendations may be presented.

In yet another example, the issue may be determined to be related to food, (e.g., calorie and/or carbohydrate intake) and thus prompt 408 may inquire whether the user ate recently and based on this response may either ask more questions, or present actionable insight and/or recommendations may be made. It is understood that prompts 404, 406, and 408 are exemplary and other troubleshooting prompts may be generated and/or displayed on a mobile device, smart device, insulin pump, or other user device.

Referring now to FIG. 4B, exemplary user interfaces including prompts for collecting food data are illustrated. As shown in FIG. 4B, prompts may include positive reinforcement and may even be generated when no issue is detected. For example, prompt 412 may be a status screen which may indicate operation and/or physiological status. Prompt 412 may be a home screen for example. The text may change when an issue is detected.

Prompt 412 may be caused to be presented on a mobile device, smart device, insulin pump, and/or other user device, and may indicate that everything is good with pump operation and/or blood glucose levels and may indicate that glucose has been in range and stable and is expected to stay that way. Prompt 412 may also include an inquiry and/or option to add food, which may be engaged by the user to add information about food or drink consumed or about to be consumed.

When the user indicates that they would like to add food, prompt 414 may be generated, giving the user the option of either confirming a meal or choosing a size of a meal. In one example, the user may need only select “add food” or “confirm” to indicate a meal for a given time. If the user selects confirm, a timestamp at that time may be generated and associated with a meal consumed at that time. The meal may be assumed to be an average meal size unless “choose size” is selected.

If “choose size” is selected prompt 416 may then permit the user to select a typical or standard meal size or small or large, which may be determined have more calories and/or carbohydrates than the standard meal size. Alternatively, prompt 418 may be generated which may have a button or engagable feature for typical meal size and a sliding scale for adjusting the meal size between less and more. It is understood that prompts could be visually presented on a display and/or audibly presented and/or answers may be tactical, typed, and/or audible.

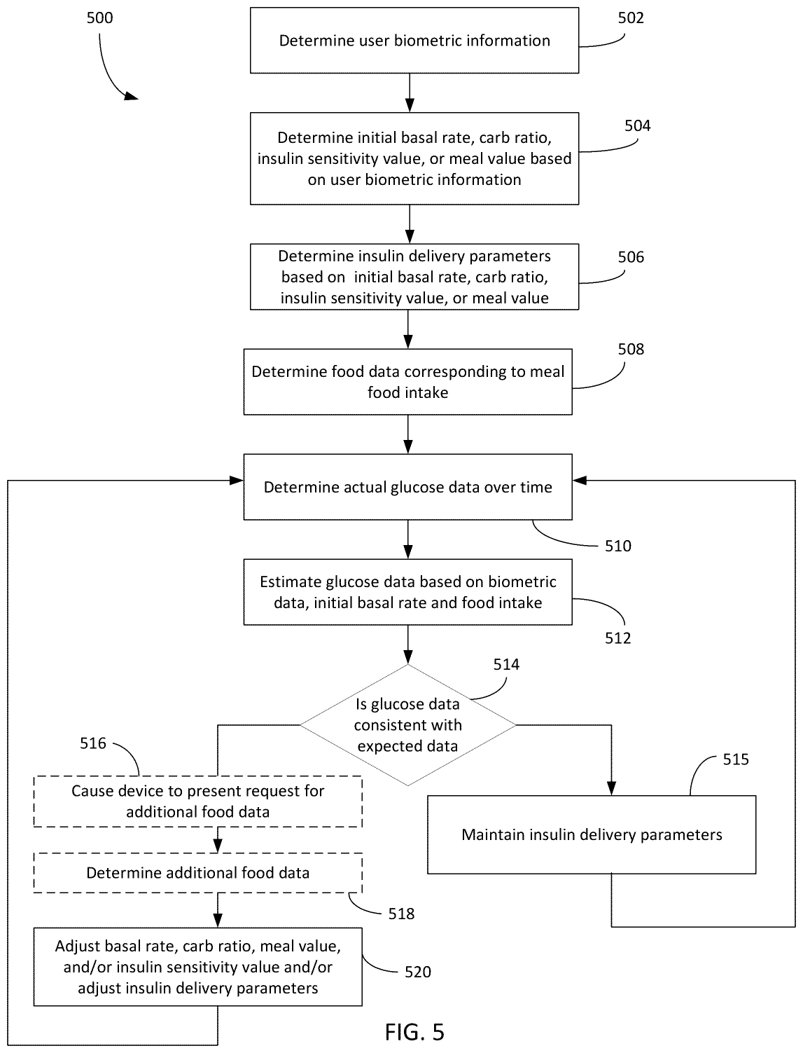

Referring now to FIG. 5, an exemplary data flow for comparing glucose levels to expected levels and improving blood glucose level outcomes based on food data is depicted. One or more steps of process flow 500 may be performed by a remote device (e.g., remote device 106 of FIG. 1) and/or a periphery device (e.g., mobile device 104 and/or smart device 108 of FIG. 1). It is further understood that one or more steps in FIG. 5 may be optional and/or additional or fewer steps than those shown in FIG. 5 may be performed.

To initiate process flow 500, at block 502, computer-executable instructions stored on a memory of a device, such as a remote device and/or a periphery device, may be executed to determine user biometric information (e.g., biometric data) which may include information about the user such as height, weight, age, sex, for example. Other information about the user such as health information, medical history, medications, and the like may also be determined.

At block 504, computer-executable instructions stored on a memory of a device, such as a remote device and/or a periphery device, may be executed to determine parameters for determining a proper amount (e.g., dose) and frequency of insulin for insulin delivery, such as an initial basal rate, carb ratio, inulin sensitivity value, and/or the like based on the biometric and other data determined. An initial meal value representative of a caloric and/or carbohydrate value for a typical meal may be determined based on the initial basal rate, carb ratio, inulin sensitivity values and/or the biometric data.

At block 506, computer-executable instructions stored on a memory of a device, such as a remote device and/or a periphery device, may be executed to determine initial insulin delivery parameters for periodically delivering insulin to the user at certain doses based on the an initial basal rate, carb ratio, inulin sensitivity value and/or meal value.

After determining initial insulin delivery parameters, at block 508, computer-executable instructions stored on a memory of a device, such as a remote device and/or a periphery device, may be executed to determine food data corresponding to food (e.g., food or drink) intake or consumption by the user. Food data may be associated with specific times (e.g., timestamps) and may indicate that a meal occurred at or around that time and may even include a size for the given meal.

At block 510, computer-executable instructions stored on a memory of a device, such as a remote device and/or a periphery device, may be executed to determine glucose data (e.g., blood glucose measurements) over time (e.g., from a glucose monitor). This data may be referred to actual glucose data. At block 512 computer-executable instructions stored on a memory of a device, such as a remote device and/or a periphery device, may be executed to estimate glucose data such as a glucose level, range, and/or trend over of time, referred to as estimated glucose data.

At decision 514, computer-executable instructions stored on a memory of a device, such as a remote device and/or a periphery device, may be executed to determine whether actual glucose data is consistent with or otherwise satisfies the estimated glucose data. To be consistent with or satisfy the estimated glucose data, the actual data may be within a range of the estimated value (e.g., defined by upper and lower threshold values) for a certain period of time or percentage of time. The period of time may be a set period of time or percentage of time. If actual glucose data is consistent with the estimated glucose data, then at block 515, computer-executable instructions stored on a memory of a device, such as a remote device and/or a periphery device, may be executed to maintain insulin delivery parameters.

Alternatively, if actual glucose data is inconsistent with the estimated glucose values, then at optional block, at block 516 computer-executable instructions stored on a memory of a device, such as a remote device and/or a periphery device, may be executed to cause a periphery device to present a request for additional food data. At optional block 518, computer-executable instructions stored on a memory of a device, such as a remote device and/or a periphery device, may be executed to determine additional food data (e.g., from the periphery device).

At block 520, computer-executable instructions stored on a memory of a device, such as a remote device and/or a periphery device, may be executed to adjust the initial basal rate, carb ratio, meal value and/or insulin sensitivity value based on the food data, the additional food data, estimated glucose data and/or actual glucose data to determine an adjusted basal rate, carb ratio, meal value and/or insulin sensitivity value.

Based on the adjusted basal rate, carb ratio, meal value and/or insulin sensitivity value and/or the food data, the additional food data, estimated glucose data and/or actual glucose data, the initial insulin delivery parameters for delivering insulin may be adjusted resulting in adjusted delivery parameters. After blocks 516 and/or 515, block 510 may be reinitiated. For example, after adjusting insulin delivery parameters (e.g., pump parameters) at block 520, the actual glucose data determined at block 510 may be indicative of improved glucose levels as compared to the glucose data determined before block 520.

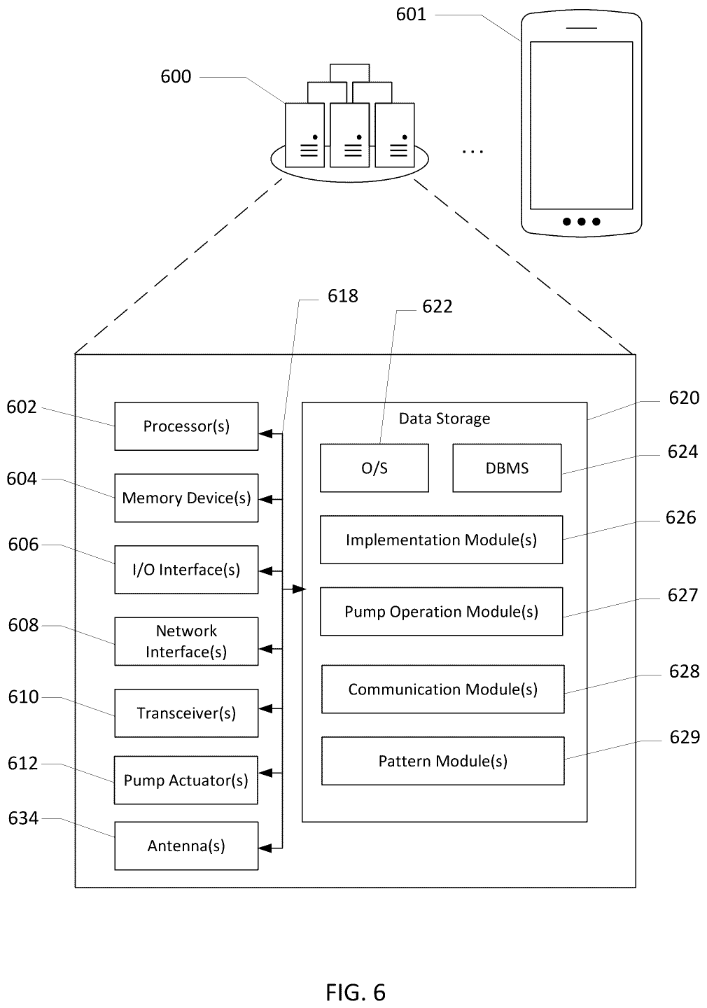

Referring now to FIG. 6, a schematic block diagram of remote device 600, which may be in communication with one or more periphery devices, glucose monitors, insulin pumps, and/or remote devices, is illustrated. Remote device 600 may be the same or similar to remote device 106 of FIG. 1 or otherwise one or more of the remote devices of FIGS. 2-5B. It is understood that an remote device 600 may alone or together with one or periphery devices and/or the insulin pump perform one or more of the operations of remote device 600 in FIG. 6. Alternatively, mobile device 601, which may be the same as or similar to mobile device 104, may perform the tasks and/or operations of remote device 600.

Remote device 600 may be designed to communicate with one or more periphery devices, remote devices, insulin pumps, smart devices, computing devices, servers, other systems, or the like. Remote device 600 may be designed to communicate via one or more networks. Such network(s) may include, but are not limited to, any one or more different types of communications networks such as, for example, near field communication networks, cable networks, public networks (e.g., the Internet), private networks (e.g., frame-relay networks), wireless networks, cellular networks, telephone networks (e.g., a public switched telephone network), or any other suitable private or public packet-switched or circuit-switched networks.

In an illustrative configuration, remote device 600 may include one or more processors 602, one or more memory devices 604 (also referred to herein as memory 604), one or more input/output (I/O) interface(s) 606, one or more network interface(s) 608, one or more transceiver(s) 610, one or more pump actuator(s) 612, one or more antenna(s) 634, and data storage 620. Remote device 600 may further include one or more bus(es) 618 that functionally couple various components of the remote device 600.

The bus(es) 618 may include at least one of a system bus, a memory bus, an address bus, or a message bus, and may permit exchange of information (e.g., data (including computer-executable code), signaling, etc.) between various components of the remote device 600. The bus(es) 618 may include, without limitation, a memory bus or a memory controller, a peripheral bus, an accelerated graphics port, and so forth. The bus(es) 618 may be associated with any suitable bus architecture including.

The memory 604 may include volatile memory (memory that maintains its state when supplied with power) such as random access memory (RAM) and/or non-volatile memory (memory that maintains its state even when not supplied with power) such as read-only memory (ROM), flash memory, ferroelectric RAM (FRAM), and so forth. Persistent data storage, as that term is used herein, may include non-volatile memory. In various implementations, the memory 604 may include multiple different types of memory such as various types of static random access memory (SRAM), various types of dynamic random access memory (DRAM), various types of unalterable ROM, and/or writeable variants of ROM such as electrically erasable programmable read-only memory (EEPROM), flash memory, and so forth.

The data storage 620 may include removable storage and/or non-removable storage including, but not limited to, magnetic storage, optical disk storage, and/or tape storage. The data storage 620 may provide non-volatile storage of computer-executable instructions and other data. The memory 604 and the data storage 620, removable and/or non-removable, are examples of computer-readable storage media (CRSM) as that term is used herein. The data storage 620 may store computer-executable code, instructions, or the like that may be loadable into the memory 604 and executable by the processor(s) 602 to cause the processor(s) 602 to perform or initiate various operations. The data storage 620 may additionally store data that may be copied to memory 604 for use by the processor(s) 602 during the execution of the computer-executable instructions. Moreover, output data generated as a result of execution of the computer-executable instructions by the processor(s) 602 may be stored initially in memory 604, and may ultimately be copied to data storage 620 for non-volatile storage.

The data storage 620 may store one or more operating systems (O/S) 622; one or more optional database management systems (DBMS) 624; and one or more program module(s), applications, engines, computer-executable code, scripts, or the like such as, for example, one or more implementation modules 626, one or more pump operation modules 627, one or more communication modules 628, and/or one more pattern modules 629. Some or all of these modules may be sub-modules. Any of the components depicted as being stored in data storage 620 may include any combination of software, firmware, and/or hardware. The software and/or firmware may include computer-executable code, instructions, or the like that may be loaded into the memory 604 for execution by one or more of the processor(s) 602. Any of the components depicted as being stored in data storage 620 may support functionality described in reference to correspondingly named components earlier in this disclosure.

Referring now to other illustrative components depicted as being stored in data storage 620, O/S 622 may be loaded from data storage 620 into memory 604 and may provide an interface between other application software executing on remote device 600 and hardware resources of the remote device 600. More specifically, O/S 622 may include a set of computer-executable instructions for managing hardware resources of remote device 600 and for providing common services to other application programs (e.g., managing memory allocation among various application programs). In certain example embodiments, O/S 622 may control execution of the other program module(s) for content rendering. O/S 622 may include any operating system now known or which may be developed in the future including, but not limited to, any server operating system, any mainframe operating system, or any other proprietary or non-proprietary operating system.

Optional DBMS 624 may be loaded into the memory 604 and may support functionality for accessing, retrieving, storing, and/or manipulating data stored in memory 604 and/or data stored in data storage 620. DBMS 624 may use any of a variety of database models (e.g., relational model, object model, etc.) and may support any of a variety of query languages. DBMS 624 may access data represented in one or more data schemas and stored in any suitable data repository including, but not limited to, databases (e.g., relational, object-oriented, etc.), file systems, flat files, distributed datastores in which data is stored on more than one node of a computer network, peer-to-peer network datastores, or the like.

I/O interface(s) 606 may facilitate the receipt of input information by remote device 600 from one or more I/O devices as well as the output of information from remote device 600 to the one or more I/O devices. The I/O devices may include any of a variety of components such as a touchscreen display; an audio output device for producing sound, such as a speaker; an audio capture device, such as a microphone; buttons and/or dials; and so forth. Any of these components may be integrated into remote device 600 or may be separate.

Remote device 600 may further include one or more network interface(s) 608 via which remote device 600 may communicate with any of a variety of other systems, platforms, networks, devices, and so forth. Network interface(s) 608 may enable communication, for example, with one or more wireless routers, one or more host servers, one or more web servers, and the like via one or more of networks.

Antenna(s) 634 may include any suitable type of antenna depending, for example, on the communications protocols used to transmit or receive signals via antenna(s) 634. Non-limiting examples of suitable antennas may include directional antennas, non-directional antennas, dipole antennas, folded dipole antennas, patch antennas, multiple-input multiple-output (MIMO) antennas, or the like. Antenna(s) 634 may be communicatively coupled to one or more transceivers 610 or radio components to which or from which signals may be transmitted or received. Antenna(s) 634 may include, without limitation, a cellular antenna for transmitting or receiving signals to/from a cellular network infrastructure, an antenna for transmitting or receiving Wi-Fi signals to/from an access point (AP), a Global Navigation Satellite System (GNSS) antenna for receiving GNSS signals from a GNSS satellite, a Bluetooth antenna for transmitting or receiving Bluetooth signals including BLE signals, a Near Field Communication (NFC) antenna for transmitting or receiving NFC signals, a 900 MHz antenna, and so forth.

Transceiver(s) 610 may include any suitable radio component(s) for, in cooperation with the antenna(s) 634, transmitting or receiving radio frequency (RF) signals in the bandwidth and/or channels corresponding to the communications protocols utilized by remote device 600 to communicate with other devices. Transceiver(s) 610 may include hardware, software, and/or firmware for modulating, transmitting, or receiving-potentially in cooperation with any of antenna(s) 634—communications signals according to any of the communications protocols discussed above including, but not limited to, one or more Wi-Fi and/or Wi-Fi direct protocols, as standardized by the IEEE 802.11 standards, one or more non-Wi-Fi protocols, or one or more cellular communications protocols or standards. Transceiver(s) 610 may further include hardware, firmware, or software for receiving GNSS signals. Transceiver(s) 610 may include any known receiver and baseband suitable for communicating via the communications protocols utilized by the remote device 600. The transceiver(s) 610 may further include a low noise amplifier (LNA), additional signal amplifiers, an analog-to-digital (A/D) converter, one or more buffers, a digital baseband, or the like.

Referring now to functionality supported by the various program module(s) depicted in FIG. 6, implementation module(s) 626 may include computer-executable instructions, code, or the like that responsive to execution by one or more of the processor(s) 602 may perform functions including, but not limited to, overseeing coordination and interaction between one or more modules and computer executable instructions in data storage 620, determining user actions, determining actions associated with user interactions, determining actions associated with user input, initiating commands locally or at periphery and/or remote devices, and the like.

Pump operation module(s) 627 may include computer-executable instructions, code, or the like that responsive to execution by one or more of the processor(s) 602 may perform functions including, but not limited to, overseeing, monitoring, and/or operating the pump and pump actuators for select delivery of a volume of insulin.

Communication module(s) 628 may include computer-executable instructions, code, or the like that responsive to execution by one or more of the processor(s) 602 may perform functions including, but not limited to, communicating with one or more devices, for example, via wired or wireless communication, communicating with mobile devices, smart devices, remote devices, charging devices, computing devices, smart sensors and/or any other devices.

The pattern module(s) 629 may include computer-executable instructions, code, or the like that responsive to execution by one or more of the processor(s) 602 may perform functions including, but not limited to, determining patterns, trends, anomalies, and/or abnormalities in the physiological, operational, and/or behavioral data, prompting users for more information based on the patterns, trends, anomalies, and/or abnormalities and/or adjusting pump operation and/or insulin delivery based on such patterns, trends, anomalies, and/or abnormalities.

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Certain aspects of the disclosure are described above with reference to block and flow diagrams of systems, methods, apparatuses, and/or computer program products according to example embodiments. It will be understood that one or more blocks of the block diagrams and flow diagrams, and combinations of blocks in the block diagrams and the flow diagrams, respectively, may be implemented by execution of computer-executable program instructions. Likewise, some blocks of the block diagrams and flow diagrams may not necessarily need to be performed in the order presented, or may not necessarily need to be performed at all, according to some embodiments. Further, additional components and/or operations beyond those depicted in blocks of the block and/or flow diagrams may be present in certain embodiments.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, may be implemented by special-purpose, hardware-based computer systems that perform the specified functions, elements or steps, or combinations of special-purpose hardware and computer instructions.

Program module(s), applications, or the like disclosed herein may include one or more software components, including, for example, software objects, methods, data structures, or the like. Each such software component may include computer-executable instructions that, responsive to execution, cause at least a portion of the functionality described herein (e.g., one or more operations of the illustrative methods described herein) to be performed.

A software component may be coded in any of a variety of programming languages. An illustrative programming language may be a lower-level programming language such as an assembly language associated with a particular hardware architecture and/or operating system platform. A software component including assembly language instructions may require conversion into executable machine code by an assembler prior to execution by the hardware architecture and/or platform.

Another example programming language may be a higher-level programming language that may be portable across multiple architectures. A software component including higher-level programming language instructions may require conversion to an intermediate representation by an interpreter or a compiler prior to execution.

Other examples of programming languages include, but are not limited to, a macro language, a shell or command language, a job control language, a script language, a database query or search language, or a report writing language. In one or more example embodiments, a software component including instructions in one of the foregoing examples of programming languages may be executed directly by an operating system or other software component without having to be first transformed into another form.

A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

Software components may invoke or be invoked by other software components through any of a wide variety of mechanisms. Invoked or invoking software components may comprise other custom-developed application software, operating system functionality (e.g., device drivers, data storage (e.g., file management) routines, other common routines, and services, etc.), or third-party software components (e.g., middleware, encryption, or other security software, database management software, file transfer or other network communication software, mathematical or statistical software, image processing software, and format translation software).

Software components associated with a particular solution or system may reside and be executed on a single platform or may be distributed across multiple platforms. The multiple platforms may be associated with more than one hardware vendor, underlying chip technology, or operating system. Furthermore, software components associated with a particular solution or system may be initially written in one or more programming languages, but may invoke software components written in another programming language.

Computer-executable program instructions may be loaded onto a special-purpose computer or other particular machine, a processor, or other programmable data processing apparatus to produce a particular machine, such that execution of the instructions on the computer, processor, or other programmable data processing apparatus causes one or more functions or operations specified in the flow diagrams to be performed. These computer program instructions may also be stored in a CRSM that upon execution may direct a computer or other programmable data processing apparatus to function in a particular manner, such that the instructions stored in the computer-readable storage medium produce an article of manufacture including instruction means that implement one or more functions or operations specified in the flow diagrams. The computer program instructions may also be loaded onto a computer or other programmable data processing apparatus to cause a series of operational elements or steps to be performed on the computer or other programmable apparatus to produce a computer-implemented process.

Additional types of CRSM that may be present in any of the devices described herein may include, but are not limited to, programmable random access memory (PRAM), SRAM, DRAM, RAM, ROM, electrically erasable programmable read-only memory (EEPROM), flash memory or other memory technology, compact disc read-only memory (CD-ROM), digital versatile disc (DVD) or other optical storage, magnetic cassettes, magnetic tape, magnetic disk storage or other magnetic storage devices, or any other medium which can be used to store the information and which can be accessed. Combinations of any of the above are also included within the scope of CRSM. Alternatively, computer-readable communication media (CRCM) may include computer-readable instructions, program module(s), or other data transmitted within a data signal, such as a carrier wave, or other transmission. However, as used herein, CRSM does not include CRCM.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

It should be understood that any of the computer operations described herein above may be implemented at least in part as computer-readable instructions stored on a computer-readable memory. It will of course be understood that the embodiments described herein are illustrative, and components may be arranged, substituted, combined, and designed in a wide variety of different configurations, all of which are contemplated and fall within the scope of this disclosure.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A method for determining patterns in an insulin delivery system to improve a user's glucose levels over time, the method comprising:
determining user data corresponding to operational information of an insulin pump of the insulin delivery system and/or user behavior information;
determining first glucose data corresponding to a set of blood glucose values of a user of the insulin pump over a first period of time;
determining an abnormality in the first glucose data based on the glucose data exceeding a threshold value for a second period of time within the first period of time;
determining an action associated with the abnormality and the user data;
causing a display to present a message indicative of the action;
determining second glucose data over a third period of time after the first period of time and after causing the display to present the message; and
determining the second glucose data satisfies the threshold value for a fourth time period during the third period of time, the second glucose data indicative of improved glucose levels for the user.

2. The method of claim 1, further comprising determining the user data corresponds to the second period of time, wherein the user data is indicative of exercise data, infusion site data, insulin delivery data, and/or food data.

3. The method of claim 1, wherein first glucose data and the second glucose data are generated by a blood glucose monitor.

4. The method of claim 1, wherein the action corresponds to eating food.

5. The method of claim 1, wherein the action corresponds to, adjusting operational settings of the insulin pump, and/or removing the insulin pump from the user.

6. The method of claim 1, wherein the second period of time is the same amount as the fourth period of time.

7. The method of claim 1, further comprising:
determining to request additional user data based on the user data and/or the abnormality; and
determining prompt information to elicit the additional user data based on the user data and/or the abnormality.

8. The method of claim 7, further comprising:
causing the display to present the prompt information; and
determining the additional user data after the causing the display to present the prompt information.

9. The method of claim 8, wherein the additional user data corresponds to additional exercise data, additional infusion site data, additional insulin delivery data, and/or additional food data.

10. The method of claim 8, wherein determining the action is based on the additional user data.

11. A system for detecting patterns relating to an insulin delivery system to improve a user's glucose levels over time, the system comprising:
memory configured to store computer-executable instructions; and
at least one computer processor configured to access memory and execute the computer-executable instructions to:
determine user data corresponding to operational information of an insulin pump of the insulin delivery system and/or user behavior information;
determine first glucose data corresponding to a set of blood glucose values of a user of the insulin pump over a first period of time;
determine an abnormality in the first glucose data based on the glucose data exceeding a threshold value for a second period of time within the first period of time;
determine an action associated with the abnormality and the user data;
cause a display to present a message indicative of the action;
determine second glucose data over a third period of time after the first period of time and after causing the display to present the message; and
determine the second glucose data satisfies the threshold value for a fourth time period during the third period of time, the second glucose data indicative of improved glucose levels for the user.

12. The system of claim 11, wherein the at least one computer processor is further configured to access memory and execute the computer-executable instructions to determine the user data corresponds to the second period of time, and wherein the user data is indicative of exercise data, infusion site data, insulin delivery data, and/or food data.

13. The system of claim 11, wherein first glucose data and the second glucose data are generated by a blood glucose monitor.

14. The system of claim 11, wherein the action corresponds to eating food.

15. The system of claim 11, wherein the action corresponds to, adjusting operational settings of the insulin pump, and/or removing the insulin pump from the user.

16. The system of claim 11, wherein the second period of time is the same amount as the fourth period of time.

17. The system of claim 11, wherein the at least one computer processor is further configured to access memory and execute the computer-executable instructions to:
determine to request additional user data based on the user data and/or the abnormality; and
determine prompt information to elicit the additional user data based on the user data and/or the abnormality.

18. The system of claim 17, wherein the at least one computer processor is further configured to access memory and execute the computer-executable instructions to:
cause the display to present the prompt information; and
determine the additional user data after the causing the display to present the prompt information.

19. The system of claim 18, wherein the additional user data corresponds to additional exercise data, additional infusion site data, additional insulin delivery data, and/or additional food data.

20. The system of claim 18, wherein determining the action is based on the additional user data.
